(19) United States
(12) Patent Application Publication
(10) Pub. No.: US 2011/0123467 A1
(43) Pub. Date: May 26, 2011

(54) COMPOSITIONS FOR PROMOTING A HEALTHY APPEARANCE OF THE SKIN

(75) Inventor: PETER T. ROTH, New York, NY (US)

(73) Assignee: Peter Thomas Roth Labs, LLC, New York, NY (US)

(21) Appl. No.: 12/848,900

(22) Filed: Aug. 2, 2010

Related U.S. Application Data
(60) Provisional application No. 61/231,948, filed on Aug. 6, 2009.

(51) Int. Cl. A61K 8/64; A61K 8/97; A61K 8/81; A61Q 17/04; A61Q 19/00

(52) U.S. Cl. 424/59; 424/771; 424/78.03; 514/18.8

(57) ABSTRACT

Topical skin care compositions are provided for the improvement of the appearance of the skin, namely of the skin comprising the face, surrounding the eyes, mouth, and hands, as well as the skin of the body. The skin care compositions include synergistic combinations of active peptide ingredients that are specially formulated to enhance the natural appearance of the face, hands, and/or body. Specifically, the synergistic skin care compositions may be applied to one or more of the skin surrounding the eyes, mouth, nose, forehead, ears, neck, hands, feed, and overall body to give the skin thereof a cleaner, smoother and more wrinkle free appearance.

### CROSS REFERENCE TO RELATED APPLICATIONS

This application claims the benefit of priority under 35 U.S.C. §119 to U.S. Provisional Patent Application Ser. No. 61/231,948, filed on Aug. 6, 2009, entitled, “Compositions For Promoting A Healthy Appearance Of The Skin”, the entire disclosure of which is incorporated by reference herein.

### BACKGROUND

Cosmetics may be used to enhance the look of a person's natural appearance. A variety of cosmetic products have been developed to promote the skin's condition and health. For instance, skin care lotions are commonly applied for the purpose of maintaining a healthy, vibrant and youthful appearance. The cosmetic industry has in large part grown to meet the needs of those seeking to make use of such products and practices by promoting and selling makeup and other skin conditioning products that are formulated to enhance the appearance of the skin. An assortment of makeup products to be applied to the body and face are available for sale. These include facial powders, creams, toners, soaps, conditioners and the like, all of which are makeup and/or cosmetic items well known in the art.

One current trend is to enhance the appearance of the skin of the face, neck, hands, and body. It is commonly thought that having clear, smooth, wrinkle-free skin surrounding the eyes, mouth, neck, hands, and body is aesthetically pleasing. Accordingly, skin care products, such as creams and lotions, are being marketed for their abilities to give a clean and smoother appearance to the skin.

However, many of the products currently sold as cosmetics have only a transitory effect that is limited to the top layers of the skin, i.e., the stratum corneum. Such cosmetics are meant to be applied and then washed off at a later time. Accordingly, although their use may result in improvement of the appearance, their efficacy is severely limited by the short duration of their effectiveness. Another limitation is that the addition of a cosmetic, if not applied properly, can give a user an unnatural and/or artificial appearance. Current trends in fashion promote a more natural, as opposed to an artificial, appearance. As a result, appearance enhancing products that are designed to promote the body's natural ability to replenish and appear more healthy are highly sought after.

There is, therefore, an interest in developing cosmetic compositions that provide a more natural, healthy and vibrant appearance of the skin and body that is longer lasting, that do not suffer from smearing, and do not give an artificial appearance when used. The compositions and methods of the present disclosure meet these and other needs. Specifically, herein are provided novel skin care compositions that reverse and/or prevent the damaging effects of free radicals. These compositions also cleanse and rejuvenate the skin, and promote and prolong a more healthy, youthful appearance.

### SUMMARY OF THE DISCLOSURE

Topical skin care compositions are provided for the improvement of the appearance of the skin, e.g., skin comprising the face, surrounding the eyes, mouth, neck, and hands, as well as the skin of the body. The skin care compositions include synergistic combinations of active ingredients that are specially formulated to enhance the natural appearance of the face, neck, hands, and/or body. The synergistic skin care compositions may be applied to one or more of the skin surrounding the eyes, mouth, lips, nose, forehead, ears, neck, hands, feet, and overall body to give the skin thereof a cleaner, healthier, smoother, and more wrinkle free appearance.

The topical skin care compositions of the disclosure include synergistic combinations of one or more natural and/or synthetic peptides or proteins, such as one or more of a peptide capable of promoting the differentiation and/or proliferation of keratinocytes and/or fibroblasts present in the tissues of the skin. In certain embodiments, a topical skin care composition of the disclosure includes a synergistic combination of one or more peptides capable of strengthening and/or rejuvenating the neuromuscular junction present in the layers of the skin. For instance, a composition of the disclosure includes one or more of a hexapeptide-19, a hexapeptide-25, a proglutamylamidoethyl indole and/or a glutamylamidoethyl indole. In certain embodiments, a composition of the disclosure includes one or more appearance enhancing factors, such as one or more of a quercus suber bark extract, dimethyl chromanyl palmitate, and/or silica in addition with DMPA/Isophthalic acid/SMDI copolymer.

In certain embodiments, a composition of the disclosure includes one or more of all of the above peptides and/or appearance enhancing factors. For example, in certain embodiments, a composition of the disclosure includes a synergistic combination of one or more, such as all of: a hexapeptide-10, an octapeptide 1, an acetyl dipeptide-1 cetyl ester, a hexapeptide-19, a hexapeptide-25, a proglutamylamidoethyl indole and/or a glutamylamidoethyl indole, a quercus suber bark extract, dimethyl chromanyl palmitate, and/or silica in addition with DMPA/Isophthalic acid/SMDI copolymer, which composition may be specially formulated to synergistically rejuvenate and condition the skin, as well as to combat the visible effects of aging. A topical skin care composition of the disclosure includes from about 0.001 to about 20% by weight hexapeptide-10, from about 0.001 to about 20% by weight octapeptide 1, from about 0.001 to about 20% by weight acetyl dipeptide-1 cetyl ester, from about 0.001 to about 20% by weight hexapeptide-19, from about 0.001 to about 20% by weight hexapeptide-25,from about 0.001 to about 20% by weight proglutamylamidoethyl indole and/or a glutamylamidoethyl indole, from about 0.001 to about 20% by weight quercus suber bark extract, from about 0.001 to about 20% by weight dimethyl chromanyl palmitate, and/or from about 0.001 to about 20% by weight silica in addition with DMPA/Isophthalic acid/SMDI copolymer.

A topical skin care composition of the disclosure may be formulated for application to the skin, such as the skin surrounding or comprising the eyes, mouth, nose, forehead, ears, neck, hands, feet, and/or overall body. For instance, the topical skin care composition may be formulated as a solution, serum, cream, lotion, body milk, emulsion, balm, gel, soap, conditioner, toner, and the like. For example, the composition may be formulated as a serum, cream, or balm and formulated for application to the skin around the eyes and/or mouth, to the eyelids, eyebrows, nose, lips, ears, neck, hands, feet, fingers, toes, and the like. In certain embodiments, the skin care composition may be formulated as a powder, e.g., for use as a foundation or other makeup component, and/or application to the face, neck, hands, and/or body.

Accordingly, the present disclosure features cosmetic skin care compositions that include one or a combination of natural and/or synthetic protein and/or peptide ingredients as well as appearance enhancing factors that function in a synergistic manner to rejuvenate, replenish, and condition the skin as well as to reduce the appearance of wrinkles therein. In certain variations, the protein, peptide, and/or appearance enhancing factor containing compositions include one or more cosmetically acceptable vehicle(s) so as to produce formulations that may be applied topically to the skin in such a manner as to moisturize the surface of the skin, soothe the muscles underlying the surface of the skin, reduce skin irritation and/or inflammation, as well as to promote a more vitalized, healthy, and youthful appearance. The cosmetic formulations of the disclosure may further be combined with one or more vasodilators, anti-inflammatories, or analgesics and/or may additionally include other skin soothing and moisturizing agents.

### DETAILED DESCRIPTION OF THE EMBODIMENTS

The skin is one of the largest organs of the body. In many instances, as a person ages the cellular and/or neuromuscular components of the skin change, which change results in a consequent change in the overall appearance of the skin. There are three main factors that affect the composition of the skin and therefore its appearance; these include: the environment, age-related cellular breakdown, and the extent of repetitive contraction of the muscles underlying the skin. For instance, many portions of the skin are continuously exposed to the environment and thus subject to the damaging effects of various environmental factors that are present in the atmosphere. For example, the skin is often exposed to harmful oxidants, such as UV radiation and other reactive oxygen species, as well as pollutants, harmful chemical agents, microbial, bacterial, and/or viral agents that are present within the environment and which can have damaging effects on the components of the skin that provide for its structural integrity and resiliency. More specifically, in certain instances, prolonged exposure to UV light, as well as other harmful environmental factors, may lead to the breakdown and/or degradation of proteins that function to hold the various layers of the skin together in a cohesive manner.

For instance, the skin is composed of several layers. Youthful skin is typically pliable, resilient, and thereby poses a stringent barrier to the harmful effects of the environment. However, as a person ages and/or with prolonged exposure to damaging environmental factors the components of the skin breakdown resulting in these layers becoming hardened, flax, and more susceptible to the environment. This breakdown in the cellular-structural components of the skin can cause the appearance of aging, wrinkles, and/or discoloration in an otherwise youthful, smooth portion of skin. The appearance of wrinkles can become even more pronounced in those individuals that are very expressive. For instance, repetitive contractile movements, such as smiling, grimacing, and the like, may cause wrinkles in the skin surrounding the portions of the body that are subjected to such repetitive movements, e.g., the skin surrounding the eyes and mouth.

The two main layers of the skin are the epidermis and dermis. The dermis lies below the epidermis and is comprised of a number of pores and structural components, such as blood vessels, nerves, hair, follicles, smooth muscles, glands, connective tissues and lymphatic tissues. A basement membrane separates the dermis from the epidermis.

The epidermis itself is composed of several layers of epithelium. The outermost layer of the epidermis of the skin is made up of a dense sub-layer of skin called the stratum corneum. The stratum corneum includes about 15 to 20 cell layers that are connected by a small number of adhesive plates, e.g., protein-rich parts of the cell membrane, which give the epidermis both a brick and mortar appearance as well as function. Between the adhesive plates of the stratum corneum are epidermal lipids, which fill the spaces between the cells like mortar or cement, and form a skin-specific lipid barrier in the structure of lamellar bilayer-lipid membranes. The epidermal lipid membranes include about 10 to 30 percent of the total volume of the stratum corneum and pose a permeability barrier which regulates the flow of water and compounds through the skin. The basement membrane is a thin sheet of fibers that underlies the epithelium of the epidermis. The basement membrane includes two basal laminae, the lamina densa and the reticular collagen fibrils and fibroblasts. The basement membrane functions to anchor the epithelium to the connective tissue beneath the epidermis and/or dermis. The dermis lies beneath the epidermis and separates the epidermis from the body's various subcutaneous tissues. The dermis is composed of two layers: the papillary dermis and the reticular dermis. The structural components of the dermis include collagen, elastic fibers, and the extrafibrillar matrix. Typically, youthful skin has a high degree of keratinization within cells of the stratum corneum as well as an elevated number of fibroblast, laminin, and collagen within the basal lamina of the basement membrane and dermis. Together these structures form a dense packed and cemented configuration of ordered, matrix that creates a firm and substantially impermeable barrier to penetration by damaging environmental agents.

However, with prolonged exposure to adverse environmental agents and/or with the general effects of aging, these proteins, epidermal lipids, nerve and/or muscle cells may become degraded and the interstitial fluid between the cells of the skin may decrease in volume leading to a breakdown in the “brick and mortar” configuration of the skin, which in turn, may result in a loss of skin elasticity and the appearance of wrinkles and/or age spots. For instance, with age and exposure to the environment, the keratinocytes, fibroblast, laminin, and/or nerve cells present in the various layers of the skin become degraded, their regeneration slows, and their functionality is compromised. When this happens the extracellular matrix surrounding the various cells of the skin breaks down and/or neurotransmission slows. In such instances, the function of the permeability barrier to both prevent the passage of various substances such as microorganisms, chemicals and allergens, and to minimize transepidermal water loss may be compromised, and thus, with exposure and aging, the skin may lose its ability to protect the body from infection and dehydration. Such a compromise may lead to skin that appears rough, old, and wrinkled.

Additionally, although the permeability barrier functions to prevent invasion from foreign molecules, it also functions to prevent and slow the penetration of chemical compounds, such as rejuvenating proteins, peptides, and their analogs, and thereby greatly reduces the effectiveness of cosmetic compositions that employ such proteins and/or their analogs. The longer it takes for an active agent, such as a protein, peptide, and/or its analog, from reaching a site of activity within the layers of the skin, the greater the chances are that the active agent will be broken down and degraded before it has the opportunity to promote its rejuvenating effects within the structures of the skin.

Another barrier to be overcome in the successful infiltration of an active agent, such as a protein and/or peptide composition of the disclosure, into the epidermis and/or into the dermis of the skin is the pH barrier thereof. The effective topical application of many active agents within cosmetic compositions currently available can be difficult because of the insolubility of the active agent(s) in various carrier compositions (such as water, alcohol or oil based carriers) and/or because of the effects that changes in the pH of the skin cause with respect to the permeability barrier of the epidermis. For example, the skin of the face produces a fine oily film that is secreted on to the surface of the skin and has a slightly acidic pH that functions as an integral part of the barrier function of the stratum corneum. The buffer capacity of the skin is the ability of healthy, normal skin to resist a change in pH from acid to alkaline, and the speed at which the skin reacts to an alkaline substance and returns to its natural acid pH value. Youthful skin of the face has a high buffer capacity that actively resists changes in pH. However, with age and/or prolonged exposure to harmful environmental factors the skin may lose its buffer capacity, resulting in increased changes to the pH level of the skin. Changes to the pH level of the skin can cause inflammation, redness and discomfort, all of which should be avoided in a cosmetic composition. Although this pH barrier protects the skin against infection, it also makes the skin less permeable to various agents of the disclosure, especially those active agents that are basic or otherwise polar compounds.

The compositions of the disclosure include novel and synergistic combinations of proteins, peptides, their analogs, and/or appearance enhancing factors as active agent(s) that are specially formulated to rejuvenate the skin, moisturize both the dermis and epidermis, increase the resiliency of the layers of the skin, as well as to relax the underlying muscles thereof. In certain instances, a composition of the disclosure may additionally increase the effectiveness of the barrier of the stratum corneum as well as help restore the pH balance of the buffer capacity of the skin.

A result of the application of a composition of the disclosure to the skin may be more healthy, vibrant and youthful appearance. In certain instances, a benefit of the application of a composition of the disclosure to the skin of the body may be a reduction in the appearance of wrinkles, e.g., a substantial reduction in the appearance of wrinkles. By substantial reduction in the appearance of wrinkles is a skin portion that appears to evidence about a 5%, such as about a 10%, for instance, about a 15%, such as a 20%, for example, a 25%, including a 30%, a 40%, or a 50% reduction in the appearance of wrinkles as compared to a portion of skin to which a composition of the disclosure has not been applied.

Accordingly, in one aspect of the disclosure, topical skin care compositions are herein provided for the cleansing, conditioning, and/or moisturizing of the skin as well as for rejuvenating and/or preventing damage associated with disruptions caused by aging and/or harmful exposure to environmental factors (e.g., oxidative stressors) and/or other skin aggravators that may lead to the appearance of aging, e.g., wrinkles, of the skin. The skin care compositions of the disclosure include synergistic combinations of cosmetically effective amounts of various proteins, peptides, analogs thereof, as well as appearance enhancing factors and/or rejuvenating emollient ingredients that are formulated to penetrate the barrier of the skin, moisturize the epidermis and dermis, restore the stratum corneum, rejuvenate the skin's natural elasticity, modulate the endogenous pH balance, and/or prevent or reverse a variety of aging-related conditions of the skin that may lead to the appearance of aging and/or wrinkles within the skin. Accordingly, an effective amount of a topical composition of the disclosure may be used to cleanse and condition the skin as well as to repair and/or protect the skin from various conditions, such as e.g., the appearance of aging, dry skin, dermatitis, eczema, sunburn, inflammation, pruritic lesions, and other inflammatory and non-inflammatory lesions of the skin of a subject. In another embodiment, the present disclosure provides cosmetic compositions that are formulated for topical administration and applied to the skin so as to reduce the effects of oxidative stress and/or to rejuvenate the skin.

By a “cosmetically effective amount” (e.g., of a skin care composition of the disclosure) is meant a quantity of the composition provided for topical administration and at a particular dosing regimen that is sufficient to achieve a desired appearance, feel, and/or protective effect. For example, an amount that results in the prevention of or a decrease in the appearance and/or symptoms associated with an undesired condition, such as wrinkles. The amount of the composition to be administered to the subject will depend on the type and severity of the condition of the skin, the amenability of the skin to respond to the formulated agents (e.g., proteins, peptides, and/or their analogs, appearance enhancing factors, and or rejuvenating emollients), and on the characteristics of the subject and the subject's natural ability to respond to the synergistic compositions of the disclosure; such characteristics include general health, age, sex, body weight, skin condition, and tolerance to the active agents in the compositions. One of skill will be able to determine appropriate dosages depending on these and other factors.

The term “subject,” as used herein, may be a mammal, such as a human, but may also be an animal, e.g., domestic animals (e.g., dogs, cats and the like), farm animals (e.g., cows, sheep, pigs, horses and the like) and laboratory animals (e.g., rats, mice, guinea pigs and the like).

Without being held to theory, the present cosmetic compositions are based on the discovery by the inventors that the combination of the various proteins, peptides, analogs thereof, and/or appearance enhancing factors and/or emollient agents herein detailed can be formulated into cosmetic topical compositions that are effective in promoting the rejuvenation of the skin, reducing the appearance of aging, and decreasing the amount of damage caused by adverse environmental factors and/or aging, while at the same time moisturizing and conditioning the skin as compared other such compositions.

It has been found that the active agents of the disclosure are capable of interacting with the skin of the body in such a way as to increase the skin's natural resiliency, regeneration, moisture content, and self-defense mechanisms. Specifically, without being held to theory, it is believed that the compositions of the disclosure not only promote the structural integrity of the skin, they also moisturize the skin, restore the pH barrier thereof, and/or act as a barrier in and of themselves to harmful environmental factors. The end result is that a subject's skin that is contacted with the compositions of the disclosure appears to be healthier, more vibrant looking (e.g., evidenced in a decreased appearance of wrinkles), and more resilient when faced with harmful environmental aggravators and/or aging than skin that is not conditioned with the compositions of the disclosure. Accordingly, the compositions of the disclosure create an overall decrease in the environmental stress and moisture loss experienced by the skin and result in a more youthful appearance when applied topically.

Accordingly, one aspect of the present disclosure is to provide rejuvenating cosmetic compositions that include natural and/or synthetic proteins, peptides, their analogs and/or other appearance enhancing factors and/or emollients. In another aspect, the rejuvenating compositions of the disclosure may be topically applied to the skin for the rejuvenation thereof. These and other aspects of the present disclosure will be more readily apparent and understood by considering the following description.

### Components of the Cosmetic Compositions

Accordingly, one aspect of the present disclosure is to provide rejuvenating cosmetic compositions that include natural and/or synthetic proteins, peptides, their analogs, and/or appearance enhancing factors. In certain embodiments, the rejuvenating compositions of the disclosure may be topically applied to the skin. Specifically, the individual ingredients herein provided have been found to be useful alone or in a combination formulation that when applied topically to a portion of the body protects the skin from harmful environmental factors (e.g., such as those that lead to oxidative stress) while also increasing the skin's natural resilience and rejuvenating abilities. For instance, in one embodiment, the topical compositions may be applied to the skin so as to clean or soften the skin and give it a more vibrant, youthful appearance and to decrease the indicia of aging, e.g., the appearance of wrinkles, inflammation, abrasions, and lesions.

In certain embodiments, a topical skin care composition of the disclosure includes one or more of a peptide capable of promoting the differentiation and/or proliferation of keratinocytes and/or fibroblasts; one or more peptides capable of strengthening and/or rejuvenating the neuromuscular junction present in the skin; and includes one or more appearance enhancing agents. For instance, a topical skin care composition of the disclosure includes a synergistic combination of one or more peptides capable of promoting the differentiation and/or proliferation of keratinocytes and/or fibroblasts, such as one or more of a hexapeptide-10, an Myristoyl Octapeptide-1, and an acetyl dipeptide-1 cetyl ester. In certain embodiments, a topical skin care composition of the disclosure includes a synergistic combination of one or more peptides capable of strengthening and/or rejuvenating the neuromuscular junction present in the skin, such as one or more of a hexapeptide-19, hexapeptide-25, a proglutamylamidoethyl indole and/or a glutamylamidoethyl indole. In certain embodiments, a composition of the disclosure includes one or more appearance enhancing factors, such as one or more of a quercus suber bark extract, dimethyl chromanyl palmitate, and/or silica in addition with DMPA/Isophthalic acid/SMDI copolymer.

In certain embodiments, a composition of the disclosure includes one or more or all of the above peptides and/or appearance enhancing factors. For instance, a composition of the disclosure includes a synergistic combination of: a hexapeptide-10, an Myristoyl Octapeptide-1, an acetyl dipeptide-1 cetyl ester, a hexapeptide-19, a hexapeptide-25, a proglutamylamidoethyl indole and/or a glutamylamidoethyl indole, a quercus suber bark extract, a dimethyl chromanyl palmitate, and/or silica in addition with DMPA/Isophthalic acid/SMDI copolymer. The active agent(s) of the composition may be formulated in conjunction with a cosmetically acceptable vehicle so as to be applied topically to the skin to clean, rejuvenate, reduce oxidative stress, and/or generally improve the appearance of the skin. The proteins, peptides, and/or analogs found to be useful in accordance with the disclosure are set forth and described herein below.

Hexapeptide-10, commonly known as SERILISINE™ (manufactured by Lipotec, SA, Barcelona, Spain and available from Centerchem, Norwalk, Conn.), is a synthetic peptide. Hexapeptide-10 may be used individually or in combination with one or more of the peptides and/or agents described herein. Hexapeptide-10 is an oligopeptide that functions to promote the differentiation and proliferation of keratinocytes and fibroblasts within the layers of the skin. With out being held to theory, and within the context of the present disclosure, Hexapeptide-10 functions as an agent promoting the rejuvenation and revitalization of the skin by increasing dermis densification as well as facilitating cohesion between the dermis and epidermis in connection with the basement membrane. For instance, by promoting keratinocyte and fibroblast production and proliferation, Hexapeptide-10 enhances densification of both the epidermis and dermis by increasing the number of cell layers. Specifically, Hexapeptide-10 promotes the rejuvenation of the epidermis by effectuating an increase in the synthesis of keratinocytes, which reduces the degeneration of the dermo-epidermal junction caused by aging. Further, Hexapeptide-10 promotes elasticity by effectuating an increase in the synthesis of fibroblast and laminin within the basement membrane, thereby increasing skin layer adhesion and reducing the loss of contact caused by the structural and functional breakdown of the cells of the skin due to aging. Hexapeptide-10 also stimulates α6-integrin production. In this manner, Hexapeptide-10 improves skin compactness and smoothness, by increasing the number of cell layers, thereby protecting the skin from harmful environmental factors, enhancing resiliency and vibrancy of the skin, and reducing the effects of aging. In certain embodiments, Hexapeptide-10 may be formulated along with one or more of the other active agents described herein and/or in addition with a cosmetically suitable carrier, such as, e.g., water.

Myristoyl Octapeptide-1, commonly known as SymPeptide™ 239 (manufactured by Symrise, Peterboro, N.J.), is a synthetic peptide. Myristoyl Octapeptide-1 may be used individually or in combination with one or more of the peptides and/or agents described herein. Myristoyl Octapeptide-1 is an oligopeptide that functions to promote the differentiation and proliferation of fibroblasts within the layers of the skin. With out being held to theory, and within the context of the present disclosure, Myristoyl Octapeptide-1 functions as an agent promoting matrix remodeling, tissue repair, and wound healing by increasing cellular proliferation of skin cells and thereby dermis densification. For instance, by promoting fibroblast production and proliferation, Myristoyl Octapeptide-1 enhances densification of both the epidermis and dermis by increasing the number of cell layers. Specifically, Myristoyl Octapeptide-1 promotes the rejuvenation of the basement membrane and/or dermis by effectuating an increase in the synthesis of fibroblasts, which increase promotes matrix remodeling and the strengthening of the connection between the layers of the skin. In this manner, Myristoyl Octapeptide-1 improves skin compactness and smoothness by increasing the number of cell layers and strengthening the framework of the extracellular matrix, which in turn protects the skin from harmful environmental factors, enhances resiliency and vibrancy of the skin, and reduces the effects of aging. Further, it has been found that by being formulated in a composition in combination with Hexapeptide-10, Myristoyl Octapeptide-1 acts synergistically with Hexapeptide-10 to increase cellular density and thereby to further rejuvenate the skin giving an increased youthful appearance. In certain embodiments, Myristoyl Octapeptide-1 may be formulated along with one or more of the other active agents described herein and/or in addition with a cosmetically suitable carrier, such as, e.g., glycerin and/or water.

Acetyl dipeptide-1 cetyl ester, commonly known as CALMOSENSINE™ (manufactured by Sederma SA, Le Perray-en-Yvelines France, and available from Croda Inc, Edison, N.J.), is a synthetic peptide comprised of N-Acetyl-Tyrosyl-Arginyl-Hexadecyl. Acetyl dipeptide-1 cetyl ester may be used individually or in combination with one or more of the peptides and/or agents described herein. Acetyl dipeptide-1 cetyl ester is an oligopeptide that functions to promote the differentiation and proliferation of keratinocytes within the layers of the skin. With out being held to theory, and within the context of the present disclosure, Acetyl dipeptide-1 cetyl ester functions as an agent promoting epithelial regeneration, tissue repair, and wound healing by increasing cellular proliferation of skin cells. For instance, by promoting keratinocytes production and proliferation, Acetyl dipeptide-1 cetyl ester enhances the regeneration of the epidermis by increasing cell layer thickness. Further, Acetyl dipeptide-1 cetyl ester reduces the wrinkles left by repetitive facial expressions by not only effectuating an increase in the release of relaxation mediators, such as proendorphins, including β-endorphin and met-enkephalin, but also by effectuating a decrease in calcitonin gene related peptide (CGRP), thereby reducing the skin's sensitization. In this manner, Acetyl dipeptide-1 cetyl ester improves skin compactness and smoothness, by increasing the thickness of the epidermis, thereby protecting the skin from harmful environmental factors, enhancing resiliency and vibrancy of the skin, and reducing the appearance of aging. Further still, it has been found that by being formulated in a composition in combination with both Octoapeptide-1 and Hexapeptide-10, Acetyl dipeptide-1 cetyl ester acts synergistically therewith to increase cellular density and thereby to further rejuvenate the skin giving the skin an increased youthful appearance. In certain embodiments, Acetyl dipeptide-1 cetyl ester may be formulated along with one or more of the other active agents described herein and/or in addition with a cosmetically suitable carrier, such as, e.g., butylene glycol and/or water and/or laureth-3 and/or hydroxyethylcellulose.

Hexapeptide-19 is a peptide commonly known as BoNT-L™ peptide (available from M&G Cosmetics Inc, East Quoque, N.Y.). Hexapeptide-19 or Palmitoyl Hexapeptide-19 may be used individually or in combination with one or more of the peptides and/or agents described herein. Hexapeptide-19 is a synthetic peptide that the inventors recognize as being capable of relaxing the muscles underlying the skin, such as the skin of the face, thereby promoting a smoother, less wrinkled appearance. For instance, without being held to theory, it is recognized that Hexapeptide-19 may interact with the various nerve cells of the neuromuscular junction to inhibit or otherwise reduce the release of acetylcholine from the nerve cells. Specifically, acetylcholine is a neurotransmitter that facilitates the ability of the nerves of the body to communicate with the muscles thereby causing contractions. Typically, acetylcholine is produced and encapsulated in vesicles within the nerve cells and released into the extracellular milieu at the synaptic cleft. Once in the extracellular milieu, acetylcholine contacts its corresponding receptor in the surrounding muscle cells, thereby activating the muscles. However, in order for acetylcholine to be released into the extracellular milieu, the acetylcholine containing vesicles must fuse with the cell surface membrane of the nerve cell. This fusion involves the formation of a synaptosomal associated protein (SNARE) complex. The SNARE complex forms when two corresponding molecules synaptobrevin/VAMP in association with NSF and syntaxin (STNX) bind the synaptosomal associated protein (SNAP)-25. Hexapeptide-19 disrupts the formation of this complex by displacing SNAP-25 causing a conformational change in the complex and thereby preventing fusion of the acetylcholine vesicles with the plasma membrane. By preventing the vesicles from fusing with the plasma membrane Hexapeptide-19 prevents or otherwise slows the release of acetylcholine from the nerve cells. In this manner Hexapeptide-19 is capable of acting on nerve cells in the skin to reduce muscle contractions and relax the surface of the skin. Such relaxation will give the skin a smoother, less wrinkled appearance, which appearance is often correlated with youthful, healthy skin. In certain embodiments, Hexapeptide-19 may be formulated along with one or more of the other active agents described herein and/or in addition with a cosmetically suitable carrier and/or filler, such as, e.g., water and/or carbomer and/or dextran and/or acrylates copolymer and/or phenoxyethanol.

Hexapeptide-25 is employed in a composition commonly known as DD06022™ (manufactured by Lipotec, Barcelona, Spain and available from Centerchem, Norwalk, Conn.). Hexapeptide-25 or Acetyl Hexapeptide-25 may be used individually or in combination with one or more of the peptides and/or agents described herein. Hexapeptide-25 is a synthetic peptide that the inventors recognize as being capable of also relaxing the muscles underlying the skin, such as the skin of the face, thereby promoting a smoother, less wrinkled appearance. For instance, without being held to theory, it is recognized that Hexapeptide-25 may interact with the various nerve cells of the neuromuscular junction to inhibit the clustering of acetylcholine receptors. Specifically, once acetylcholine is released into the extracellular milieu from the nerve cells it contacts corresponding acetylcholine receptors in the surrounding muscle cells, thereby signaling receptor clustering and causing the muscles to contract. Hexapeptide-25, however, competes with the acetylcholine in the extracellular milieu for receptor cell binding and thus acts as an inhibitor of nerve cell transmission. In this manner Hexapeptide-25 is capable of acting at the neuromuscular junction in the skin to reduce muscle contractions and relax the surface of the skin. Such relaxation will give the skin a smoother, less rigid and more youthful appearance. Further, it has been found that by being formulated in a composition in combination with Hexapeptide-19, Hexapeptide-25 acts synergistically with Hexapeptide-19 to reduce muscle contraction and thereby to further reduce the appearance of wrinkles. In certain embodiments, Hexapeptide-25 may be formulated along with one or more of the other active agents described herein and/or in addition with a cosmetically suitable carrier and/or other components, such as, e.g., water and/or Arginine and/or Caprylyl Glycol.

Proglutamylamidoethyl indole and/or Glutamylamidoethyl indole are peptides used in compositions commonly known as Glutrapeptide™ and Glistin™, respectively (manufactured by Exsymol, SA, Monaco and available from Biosil, Allendale, N.J.). They are peptides comprised of L-pyroglutamic acid/decarboxytryptophan and L-glutamic acid/decarboxytryptophan respectively. Proglutamylamidoethyl indole and/or a Glutamylamidoethyl indole may be used individually or in combination together with one or more of the peptides and/or agents described herein. Proglutamylamidoethyl indole and/or a Glutamylamidoethyl indole are synthetic peptidomimetic nueroactive peptides that the inventors recognize as being capable of functioning as neuroprotective agents. Without being held to theory, these compounds function similar to neurotrophic growth factors that strengthen the nervous epidermic network of the skin, enhancing the interaction between the nerves, muscles, and other various cells of the skin including keratinocytes and fibroblasts. Specifically, these compounds promote differentiation of the cutaneous nerve cells and further evidence anti-apoptotic properties that protect these cells from cell death. For instance, Proglutamylamidoethyl indole and/or a Glutamylamidoethyl indole strengthen the neuronal communication network by promoting neurite outgrowth, stimulating IL-6 and VIP release, preserving neuronal cell morphology, and stimulating neuroendocrine activity. Additionally, Proglutamylamidoethyl indole and/or a Glutamylamidoethyl indole promote cell survival by increasing Bcl-2 protein production, which protein functions as a repressor of apoptotic death. Specifically, Bcl-2 blocks the release of apoptogenic cytochrome-C, decreasing the activation of caspase members, increasing activation of Nfκ-B, and promoting cell survival. These compounds also evidence strong anti-oxidant properties. For instance, these compounds have been found to protect neurons and keratinocytes from UVB damage as well as from oxidative stress. Hence, Proglutamylamidoethyl indole and/or a Glutamylamidoethyl indole stimulate cellular proliferation and enhance the integrity of the dermo-epidermic junction, as well as promote communication between the nerves and muscles associated with the skin, thereby enhancing cutaneous nerve terminal regeneration, and advancing wound healing through the strengthening of neural modulation. Further, Proglutamylamidoethyl indole and/or a Glutamylamidoethyl indole can improve epidermal regeneration and strengthen the skin barrier by activating the genes related to cell differentiation and re-epithelialization, the end result being the appearance of more healthy, thicker, and resilient skin. Further, it has been found that by being formulated in a composition in combination with Hexapeptide-19 and Hexapeptide-25, Proglutamylamidoethyl indole and/or a Glutamylamidoethyl indole act synergistically therewith to reduce muscle contraction and thereby to further reduce the appearance of wrinkles. In certain embodiments, Hexapeptide-25 may be formulated along with one or more of the other active agents described herein and/or in addition with a cosmetically suitable carrier and/or other components, such as, e.g., water and/or butylene glycol.

As set forth above, a composition of the disclosure includes one or more appearance enhancing factors, such as one or more of a quercus suber bark extract, dimethyl chromanyl palmitate, and/or silica in addition with DMPA/Isophthalic acid/SMDI copolymer, which may be used in a composition of the disclosure individually or in combination with one or more of the other peptides and/or agents described herein. For instance, a composition includes an extract from the quercus suber bark. Specifically, suberin, e.g., hydrolyzed suberin, is a natural agent extracted from the bark of the Quercus suber tree. Suberin is available as SUBERLIFT™, which is manufactured by International Specialty Products Corp., Wayne, N.J.. It has been found that Quercus Suber Bark Extract or Suberin interacts with the cells present on the surface of the skin to lift and tighten the skin thereby giving it a more taut and smooth surface appearance. In certain embodiments, Quercus Suber Bark Extract may be formulated along with one or more of the other active agents described herein and/or in addition with a cosmetically suitable carrier, such as, e.g., water and/or dipropylene glycol.

Additional skin appearance enhancing agents, such as dimethyl chromanyl palmitate and/or silica in addition with DMPA/Isophthalic acid/SMDI copolymer may also be used by themselves or in combination with one or more agents of the disclosure to give the skin a more smooth, compact and youthful appearance. For instance, dimethyl chromanyl palmitate is commonly known as CHROMOBRIGHT™ and is available in a composition with dimethiconol, ethylhexyl cocoate, and dimethylmethoxy chromanyl palmitate, which composition is available from Centerchem, Norwalk, Conn. Specifically, dimethyl chromanyl palmitate has been found to act as a skin lightening agent that is capable of depigmenting melanocytes. For example, dimethyl chromanyl palmitate may function to increase tyrosinase inhibition, thereby evidencing hypopigmentation activity; and may further have a photoprotective effect on epidermal keratinocytes, thereby preventing skin damage caused by UV radiation. Hence, dimethyl chromanyl palmitate may help diminish the appearance of skin discolorations, such as those known as sunspots. In certain embodiments, dimethyl chromanyl palmitate may be formulated along with one or more of the other active agents described herein and/or in addition with a cosmetically suitable carrier and/or other components, such as, e.g., cyclopentasiloxane and/or dimethiconol and/or edthylhexyl cocoate. Additionally, silica in addition with DMPA/Isophthalic acid/SMDI copolymer, which may be obtained in the form of microspheres commonly known as CHRONOSPHERES™, has been shown to obscure incident light contacting the surface of the skin. By obscuring the harshness of incident light, these microspheres function to hide the lines and other skin imperfections, such as those caused by wrinkles giving the skin the appearance of luminescence and smoothness. Accordingly, one or more of these appearance enhancing factors may be employed in a composition of the disclosure to enhance the appearance of the skin, e.g., the decrease the observable appearance of wrinkles.

Accordingly, in a particular embodiment, a composition of the disclosure includes as an active agent at least one or more peptide components selected from (e.g., a synergistic combination of): a hexapeptide-10, an Myristoyl Octapeptide-1, an acetyl dipeptide-1 cetyl ester, a hexapeptide-19, a hexapeptide-25, a proglutamylamidoethyl indole and/or a glutamylamidoethyl indole, a quercus suber bark extract, a dimethyl chromanyl palmitate, and/or silica in addition with DMPA/Isophthalic acid/SMDI copolymer. In certain variations, a composition of the disclosure includes as an active agent one peptide component capable of promoting the differentiation and/or proliferation of keratinocytes and/or fibroblasts, such as a peptide component selected from: a hexapeptide-10, an Myristoyl Octapeptide-1, an acetyl dipeptide-1 cetyl ester. In addition, in certain variations, a composition of the disclosure includes as an active agent one peptide component capable of strengthening and/or rejuvenating the neuromuscular junction present in the skin, such as a peptide component selected from a hexapeptide-19, a hexapeptide-25, a proglutamylamidoethyl indole and/or a glutamylamidoethyl indole. In further addition, in certain variations, a composition of the disclosure includes as an active agent a skin appearance enhancing factor selected from a quercus suber bark extract, a dimethyl chromanyl palmitate, and silica in addition with DMPA/Isophthalic acid/SMDI copolymer. In certain variations, a composition of the disclosure includes as an active agent two peptide components capable of promoting the differentiation and/or proliferation of keratinocytes and/or fibroblasts, such as two peptide components selected from: a hexapeptide-10, an Myristoyl Octapeptide-1, an acetyl dipeptide-1 cetyl ester. In addition, in certain variations, a composition of the disclosure includes as an active agent two peptide components capable of strengthening and/or rejuvenating the neuromuscular junction present in the skin, such as two peptide components selected from a hexapeptide-19, a hexapeptide-25, a proglutamylamidoethyl indole and/or a glutamylamidoethyl indole. In addition, in certain variations, a composition of the disclosure includes as an active agent two skin appearance enhancing factors selected from a quercus suber bark extract, a dimethyl chromanyl palmitate, and silica in addition with DMPA/Isophthalic acid/SMDI copolymer. In certain variations, a composition of the disclosure includes as an active agent all of a hexapeptide-10, an Myristoyl Octapeptide-1, an acetyl dipeptide-1 cetyl ester, a hexapeptide-19, a hexapeptide-25, a proglutamylamidoethyl indole and/or a glutamylamidoethyl indole, a quercus suber bark extract, a dimethyl chromanyl palmitate, and/or silica in addition with DMPA/Isophthalic acid/SMDI copolymer.

The cosmetic compositions of the present disclosure are formulated for topical use. Accordingly, in a particular embodiment, a component from the one or more peptides and/or agents detailed herein may serve as an active agent and may be incorporated into a topical composition by itself or in combination with one or more of the other active agents. In general, the subject cosmetic compositions contain at least about 0.0001%, at least about 0.01%, at least about 0.1%, or at least about 1.0% and may be as great as or more than about 5% or about 10% or about 15%, or about 20%, or about 25% or more (weight/weight). For instance, in certain variations, a composition of the disclosure may contain from about 0.0001% to about 20% of a peptide selected from one or more of a hexapeptide-10, an Myristoyl Octapeptide-1, an acetyl dipeptide-1 cetyl ester, a hexapeptide-19, a hexapeptide-25, a proglutamylamidoethyl indole and/or a glutamylamidoethyl indole, a quercus suber bark extract, a dimethyl chromanyl palmitate, and/or silica in addition with DMPA/Isophthalic acid/SMDI copolymer. as an active ingredient. In certain variations, a composition of the disclosure may contain from about 0.0001% to about 20% of a peptide and/or agent selected from all of a hexapeptide-10, an Myristoyl Octapeptide-1, an acetyl dipeptide-1 cetyl ester, a hexapeptide-19, a hexapeptide-25, a proglutamylamidoethyl indole and/or a glutamylamidoethyl indole, a quercus suber bark extract, a dimethyl chromanyl palmitate, and/or silica in addition with DMPA/Isophthalic acid/SMDI copolymer.

Without being held to theory, the anti-aging and/or anti-wrinkle and/or relaxation effects of the composition of the disclosure stems in part from the synergistic activities of its individual peptide and/or appearance enhancing factor components, which may relax the muscles of the skin, promote the structuring of the epidermis and/or dermis and/or extracellular matrix, and/or counteract the oxidative effects of environmental factors, such as free radicals, for instance, reactive oxygen species, which can lead to inflamed, irritated and dry skin and/or promote the generation of other skin-damaging free-radicals. As such, the cosmetic compositions of the present disclosure are uniquely suited to rejuvenate and condition the skin. In some embodiments, the cosmetic compositions of the present disclosure are carefully formulated to provide one or more of a hexapeptide-10, an Myristoyl Octapeptide-1, an acetyl dipeptide-1 cetyl ester, a hexapeptide-19, a hexapeptide-25, a proglutamylamidoethyl indole and/or a glutamylamidoethyl indole, a quercus suber bark extract, a dimethyl chromanyl palmitate, and/or silica in addition with DMPA/Isophthalic acid/SMDI copolymer, in active form as active agents, thereby maximizing the effectiveness of the overall composition.

In many embodiments, additional ingredients are included as part of the subject cosmetic compositions to enhance the synergistic effect of the active agents of the peptide compositions. Accordingly, one or more of the following ingredients may be included in a cosmetic formulation of the present disclosure.

In certain variations, a composition of the disclosure may additionally include one or more other beneficial agents as set forth herein below.

### Skin Soothing/Conditioning Agents

The cosmetic compositions of the present disclosure may also contain agents that soothe, condition and/or rejuvenate the skin. One such agent is panthenol, a pro-vitamin moisturizing agent. Panthenol may be incorporated into cosmetic formulations and may function within a cosmetic composition of the disclosure by readily penetrating the skin to help deliver the active agents to a site of action and to sooth and moisturize the skin. Panthenol derivatives (e.g., ethyl panthenol) may also be used in the compositions of the disclosure as well as other agents such as provitamins B5 and E, as well as their derivatives, aloe vera (e.g., for soothing the skin), allantoin (e.g., for both soothing and softening of the skin), bisabolol (e.g., for soothing the skin), dipotassium glycyrrhizinate, pantothenic acid and its derivatives, as well as Sorbitol (e.g., for moisturizing the skin). A variety of emollients may also be included, such as Neopentyl Glycol Dicaprylate/Dicaprate-ester, Dimethicone, Tridecyl Trimellitate, and other such suitable emollients known in the art. Other skin conditioning/soothing agents can be included in the subject compositions, some of which are discussed below. For instance, one or more of the following may be included: Bis-PEG-18 Methyl Ether Dimethyl Silane, Squalane, PEG 3350, Phospholipids, Vitamin A and its derivatives, Sodium Hyaluronate, Behenyl Alcohol, Butyrospermum Parkii (Shea Butter), Cyclomethicone, Lactobacillus/Eriodictyon Californicum Ferment Extract and the like, Methyl Gluceth-20, Prunus amygdalus dulcin (Sweet Almond) Oil, Sodium Lactate, Borago Officinalis (Borage) Seed Oil, Persea Gratissima (Avocado) Oil, Vitamin D and its derivatives, Caprylic/Capric Triglyceride, and the like. Other beneficial agents include: Lanolin Oil, Lanolin Alcohol, Ceramide III, Urea, Trehalose, mannan, and the like. The skin soothing and/or conditioning agents may be present at a concentration of at least about 0.01% or more, including about 0.25% or more, such about 0.5% or more, for instance about 1.0% to about 10% or more, such as about 20% or more.

### Optional Skin Benefit Materials and Cosmetic Adjuncts

The compositions of the disclosure may optionally include other beneficial materials.

These include steroidiol hormones; progesterone; pregnanalone; coenzyme Q10; methylsolanomethane (MSM); sodium phytate (which may function as a chelating and/or stabilizing agent); copper peptide (copper extract); plankton extract (phytosome); transforming growth factor beta 1 (TGF-β1); glycolic acid; kojic acid; ascorbyl palmitate; all-trans-retinol; azaleic acid; salicylic acid; analgesics; non-steroidal anti-inflammatory drugs (NSAIDs); broparoestrol; and the like. If present, steroids may be present at a concentration of less than about 2% of the total by weight of the composition, while the other optional skin benefit materials may be present at higher levels, for example as much as 10 to 15%.

The compositions may further include sunscreens to lower skin's exposure to harmful UV rays. Sunscreens include those materials commonly employed to block ultraviolet light and, for example, may be found on the current FDA Monograph for Sunscreen Actives. Exemplary compounds are the derivatives of PABA, cinnamate and derivatives of salicylate (other than ferulyl salicylate). For example, octyl methoxycinnamate and 2-hydroxy-4-methoxy benzophenone (also known as oxybenzone) can be used. Octyl methoxycinnamate and 2-hydroxy-4-methoxy benzophenone are commercially available under the trademarks, Parsol MCX and Benzophenone-3, respectively. Dermascreen may also be used. Additionally, a composition may further include aloe vera or an extracted component or powder thereof, such as anthraquinone glycosides, resins, polysaccharides, sterols, gelonins, and chromones. The exact amount of sunscreen or aloe employed in the compositions can vary depending upon the degree of protection desired from the sun's UV radiation.

The cosmetic compositions of the present disclosure may also contain one or more of the following agents, which agents may act synergistically with the various active agents in various cosmetic compositions of the disclosure to treat aging-related conditions and/or disorders. Butylene Glycol, Saccharomyces Cerevisiae Extract, Tripeptide-29, Sodium Chondroitin Sulfate, Hyaluronic Acid, Silica in addition with DMPA/Isophthalic acid/SMDI copolymer Dimethyl Silylate, Yeast Polysaccharides, Glycine Soja (Soybean) Seed Extract, Lactobacillus/Capsicum Frutescens Fruit Extract, Pectin, Hydrolyzed Vegetable Protein, Serine, Arginine, Proline. Pentylene Glycol, Sodium Polyacrylate, C 12-15 Alkyl Benzoate, Matricaria recutita (Chamomile) Extract, Symphytum Officinale (Comfrey) Extract, Camellia Sinensis (Green Tea) Leaf Extract, Alanine, Ethoxydiglycol, Hydrogenated Lecithin, and the like. Other beneficial materials include Glycerin, Histidine, Collagen, Elastin, Algae Extract, Echinacea Angustifolia Leaf Extract, Ginkgo Biloba Extract, Vitis vinifera (grape) seed extract, Sodium PCA, Centella Asiatica (Gotu Kola) Extract, Wheat amino acids, Foeniculum vulgare (Fennel) Seed extract, Linoleic acid, Oleic acid, Viola tricolor extract, Rosa Canina Fruit Oil, Superoxide Dismutase, Linoleic acid, Oleic acid, Polyquaternium 51, Sodium cocoyl amino acids, Sarcosine, Potassium aspartate, Magnesium aspartate, Glycosaminoglycans, and the like.

The cosmetic compositions of the present disclosure may also contain ingredients contained in the current FDA Monograph for Acne Actives including benzoyl peroxide, which may act synergistically with the various active agents in various cosmetic compositions of the disclosure to treat aging-related conditions and/or disorders. Benzoyl peroxide is a commonly used topical treatment for mild acne. Benzoyl peroxide has antiseptic properties, i.e., it reduces the number of skin surface bacteria and yeast; is an oxidizing agent, making it keratolytic and comedolytic; and has anti-inflammatory activity. The cosmetic compositions may contain benzoyl peroxide at concentrations contain at least about 1%, at least about 2.5%, at least about 5%, and usually not more than about 10% (weight/weight). Benzoyl peroxide is commercially available as a cream, gel, lotion, or wash under the following brand names: Benoxyl™, Benzac™, Brevoxyl™, Oxy™ and PanOxyl™.

### Cosmetically Acceptable Vehicle

The compositions of the disclosure include a cosmetically acceptable vehicle to act as a dilutant, dispersant or carrier for the active ingredients, so as to facilitate its distribution and uptake when the composition is applied to the skin. Vehicles other than or in addition to water can include liquid or solid emollients, solvents, humectants, thickeners, powders, and perfumes, some of which have been described above. For instance, a suitable carrier can be glycerin, butylene glycol, propylene glycol, water, various oils (jojoba, sweet almond, soybean, sunflower, apricot, etc.), and the like.

The cosmetically acceptable vehicle may range form from about 1% or less to about 99.9% or more, such as from about 10% to about 90%, including about 25% to about 80% by weight of the composition, and can, in the absence of other cosmetic adjuncts, form the balance of the composition.

The compositions may be in the form of aqueous, aqueous/alcoholic or oily solutions; dispersions of the lotion or serum type; anhydrous or lipophilic gels; emulsions of liquid or semi-liquid consistency and/or water in silicone (W/Si) or silicone in water (Si/W), which are obtained by dispersion of a fatty phase in an aqueous phase (O/W) or conversely (W/O); or suspensions or emulsions of smooth, semi-solid or solid consistency of the cream or gel type. These compositions are formulated according to the usual techniques as are well known to this art.

When the compositions of the disclosure are formulated as an emulsion, the proportion of the fatty phase and/or silicone phase may range from about 5% or less to about 80% or more by weight, such as from about 10% to about 50% by weight, relative to the total weight of the composition. Oils, emulsifiers, co-emulsifiers, and/or silicones incorporated in the composition in emulsion form are selected from among those used conventionally in the cosmetic or dermatological field. The emulsifier and coemulsifier may be present in the composition at a proportion ranging from about 0.3% or less to about 30% or more by weight, such as from about 0.5% to about 20% by weight, relative to the total weight of the composition.

When the compositions of the disclosure are formulated as an oily solution or gel, or a silicone solution or gel, the fatty phase or silicone phase respectively may constitute more than about 50% or less, more than about 60%, more than about 70%, more than about 80%, more than about 90% of the total weight of the composition.

The compositions of the disclosure may be in the form of body cleansing compositions. As such, these compositions may contain one or more wash-active surfactant in an aqueous base. The surfactants can be present, alone or in a mixture, and are contained in an amount of from about 1% or less to about 50% or more by weight or from about 1% or less to about 30% or more by weight. Nonionic surfactants, amphoteric surfactants, zwitterionic surfactants and anionic surfactants are generally suitable.

Suitable anionic surfactants include, e.g. alkaline or alkaline earth salts, alpha-olefin sulfonates, sulfosuccinates, disodium laureth-3 sulfosuccinate, disodium PEG-5 lauryl citrate sulfosuccinate, disodium ricinolamido MEA-sulfosuccinate or disodium laurylamido MEA-sulfosuccinate, alkyl ether carboxylates, alkyl sulfate and alkyl ether sulfates.

Suitable nonionic surfactants include e.g., alkoxylated fatty alcohols, alkoxylated fatty acid esters, alkoxylated partial glycerides, saturated or unsaturated fatty acids, alkoxylated polyol esters, and alkylpolyglucosides, such as coconut glucosides, lauryl glycosides or decylglucosides. For example, ethoxylated lauryl alcohol, tetradecyl alcohol, cetyl alcohol, oleyl alcohol or stearyl alcohol, which are used alone or in mixtures with each other, as well as fatty alcohols of ethoxylated lanolin, are suitable as fatty alcohol ethoxylates. Furthermore the ethoxylated fatty acid sugar esters known as nonionic surfactants, especially ethoxylated sorbitan fatty acid ester, are suitable for use in the cosmetic preparations according to the disclosure. The suitable ethoxylated fatty acid sugar esters include those marketed under the trade names Tween™ and Arlacel™ by ICI surfactants and the alkyl-polyglycosides, which are marketed under the trade names Plantaren™ or Plantacare™ by Henkel or under the trade name Oramix™ by Seppic. Suitable amphoteric surfactants include for example betaines, such as cocoamidopropylbetaine or lauryl betaine, sulfobetaines, such as cocoamidopropyl hydroxysultaine, glycinates, such as cocoamphoglycinate (INCI-name: sodium cocoamphoacetate) and diglycinates and propionates, such as cocoampho-propionate.

The compositions of the disclosure may also include additives and adjuvants which are conventional in the cosmetic, pharmaceutical or dermatological field, such as hydrophilic or lipophilic gelling agents, hydrophilic or lipophilic active agents, preservatives, antioxidants, solvents, fragrances, perfumes, sunscreens, fillers, bactericides, odor absorbers and dyestuffs or colorants. The amounts of these various additives and adjuvants are those conventionally used in the field, and, for example, range from about 0.01% or less to about 10% or more of the total weight of the composition. Depending on their nature, these additives and adjuvants may be introduced into the fatty phase, silicone phase, or into the oil phase (which includes silicone) or into the aqueous phase.

Exemplary oils which may be used according to this disclosure include mineral oils (liquid petrolatum), plant oils (e.g., butters, such as a liquid fraction of karite butter, sunflower oil), animal oils (perhydrosqualen(e)), synthetic oils (purcellin oil), silicone oils (cyclomethicone) and fluoro oils (perfluoropolyethers). Fatty alcohols, fatty acids (stearic acid) and waxes (paraffin wax, carnauba wax and beeswax) may also be used as fats.

Emulsifiers which may be used include glyceryl stearate (e.g., to help the oil and water phases combine), polysorbate 60, PEG-6/PEG-32/glycol stearate mixture, etc. Solvents which may be used include the lower alcohols, in particular ethanol and isopropanol, and propylene glycol. Suitable fatty alcohols and acids include those compounds having from 10 to 25 carbon atoms. For instance, compounds such as cetyl, myristyl, palmitic and stearyl alcohols and acids. For example, cetearyl alcohol and ceteareth 20, as well as cetyl alcohol and stearyl alcohol-fatty alcohols may also be included.

An oil or oily material may be present, together with an emollient to provide either a water-in-oil emulsion or an oil-in-water emulsion, depending largely on the average hydrophilic-lipophilic balance (HLB) of the emollient employed. Levels of such emollients may range from about 0.5% to about 50%, preferably between about 5% and 30% by weight of the total composition. Emollients may be classified under such general chemical categories as esters, fatty acids and alcohols, polyols and hydrocarbons.

Esters may be mono- or di-esters. Acceptable examples of fatty di-esters include dibutyl adipate, diethyl sebacate, diisopropyl dimerate, and dioctyl succinate. Acceptable branched chain fatty esters include 2-ethyl-hexyl myristate, isopropyl stearate and isostearyl palmitate. Acceptable tribasic acid esters include triisopropyl trilinoleate and trilauryl citrate. Acceptable straight chain fatty esters include lauryl palmitate, myristyl lactate, oleyl eurcate and stearyl oleate. Preferred esters include coco-caprylate/caprate (a blend of coco-caprylate and coco-caprate), propylene glycol myristyl ether acetate, diisopropyl adipate and cetyl octanoate.

Among the polyols which may serve as emollients are linear and branched chain alkyl polyhydroxyl compounds. For example, propylene glycol, sorbitol and glycerin are preferred. Also useful may be polymeric polyols such as polypropylene glycol and polyethylene glycol. Butylene and propylene glycol are also especially preferred as penetration enhancers.

Exemplary hydrocarbons which may serve as emollients are those having hydrocarbon chains anywhere from 12 to 30 carbon atoms. Specific examples include mineral oil, petroleum jelly, squalene and isoparaffins.

Another category of functional ingredients within the cosmetic compositions of the present disclosure are thickeners. A thickener may be present in amounts anywhere from about 0.1% or less to about 20% or more by weight, such as from about 0.5% to about 10% by weight of the composition. Exemplary thickeners may be cross-linked polyacrylate materials available under the trademark Carbopol. Gums may be employed such as xanthan, carrageenan, gelatin, karaya, pectin and locust beans gum. Under certain circumstances the thickening function may be accomplished by a material also serving as a silicone or emollient. For instance, silicone gums in excess of 10 centistokes and esters such as glycerol stearate have dual functionality. Hydrophilic gelling agents include carboxyvinyl polymers (carbomer), acrylic copolymers, such as acrylate/alkylacrylate copolymers, polyacrylamides, polysaccharides, such as hydroxypropylcellulose, natural gums and clays (such as Xanthan gum), and, as lipophilic gelling agents, representative are the modified clays such as bentones, fatty acid metal salts such as aluminum stearates and hydrophobic silica in addition with DMPA/Isophthalic acid/SMDI copolymer, or ethylcellulose and polyethylene.

Powders may be incorporated into the cosmetic composition of the disclosure. These powders include chalk, talc, kaolin, starch, smectite clays, chemically modified magnesium aluminum silica in addition with DMPA/Isophthalic acid/SMDI copolymerte, organically modified montmorillonite clay, hydrated aluminum silica in addition with DMPA/Isophthalic acid/SMDI copolymerte, fumed silica in addition with DMPA/Isophthalic acid/SMDI copolymer, aluminum starch octenyl succinate and mixtures thereof.

Other adjunct minor components may also be incorporated into the cosmetic compositions. These ingredients includes coloring agents, opacifiers and perfumes. Amounts of these other adjunct minor components may range anywhere from about 0.001% or less up to about 20% or more by weight of the composition.

### Product Use, Form, and Packaging

In use, a quantity of the composition, for example from about 1 ml or less to about 100 ml or more, is applied to a site of interest (e.g., skin, scalp, etc.) from a suitable container or applicator and, if necessary, it is then spread over and/or rubbed into the site using the hand or fingers or a suitable device, such as a brush, comb, or other suitable applicator. The composition may be specifically formulated for use as a treatment for a specific area, e.g. the face, the skin surrounding the eyes, nose, mouth, and/or chin, the scalp, the neck, the hands, the feet, etc, such as for the conditioning and/or diminishing the appearance of forehead folds, lateral line folds, chin and nasal folds, upper lip wrinkles, platysmal folds, décolleté folds, and/or glabellar frown lines, etc. The compositions may be formulated for application to the forehead, eye zone area, nose zone area, mouth or lip zone area, the face, the hands, feet, arms, legs, body, and the like.

The cosmetic composition of the disclosure may be formulated in any form suitable for application to the site of interest, including a lotion, cream, gel, shampoo, conditioner, tightening product, toner, a serum, a foundation, etc. The composition can be packaged in a suitable container to suit its viscosity and intended use by the consumer. For example, a lotion or cream can be packaged in a bottle, or a propellant-driven aerosol device or a container fitted with a pump suitable for finger operation. When the composition is a cream, it can simply be stored in a non-deformable bottle or squeeze container, such as a tube or a lidded jar. The disclosure accordingly also provides a closed container containing a cosmetically acceptable composition as herein defined.

The following examples are put forth so as to provide those of ordinary skill in the art with a complete disclosure and description of how to make and use the subject compositions, and are not intended to limit the scope of what is regarded as the disclosure. Efforts have been made to insure accuracy with respect to the numbers used (e.g. amounts, temperature, concentrations, etc.) but some experimental errors and deviations should be allowed for. Unless otherwise indicated, parts are parts by weight, molecular weight is weight average molecular weight, temperature is in degrees centigrade, and pressure is at or near atmospheric.

### EXAMPLE 1

Example 1 illustrates topical compositions according to the present disclosure. The compositions can be processed in accordance with conventional manners well known in the art. They are suitable for cosmetic use. In particular the compositions are suitable for application to a site of interest that has an aging-related condition or disorder. Application of the cosmetic compositions will combat these conditions thereby restoring a more youthful appearance. In addition, certain of these cosmetic compositions can be used to prevent the onset of the aging-related condition or disorder.

OIL-IN-WATER EMULSION

| Ingredient | % w/w |
|---|---|
| water | QS |
| panthenol | 0-5.00% |
| aloe barbadensis leaf juice | 0-2.00% |
| sodium phytate | 0-0.50% |
| allantoin | 0-2.00% |
| Methylparaben | 0-0.50% |
| glyceryl stearate (and) PEG- | 0-5.00% |
| 100 stearate | |
| C12-15 Alkyl Benzoate | 0-5.00% |
| Glyceryl Stearate | 0-5.00% |
| Persea Gratissima (Avocado) | 0-5.00% |
| Oil | |
| Isocetyl stearate | 0-5.00% |
| tocopheryl acetate | 0-5.00% |
| cetyl alcohol | 0-5.00% |
| dimethicone | 0-5.00% |
| Butyrospermum Parkii (Shea | 0-5.00% |
| Butter) | |
| Propylene Glycol Stearate | 0-5.00% |
| Caprylic/Capric Triglyceride | 0-5.00% |
| Stearoxytrimethylsilane (and) | 0-5.00% |
| Stearyl Alcohol | |
| Hydrogenated Lecithin | 0-5.00% |
| Acetyl Hexapeptide-25 | 0-10.00% |
| Water (and) Palmitoyl | 0-10.00% |
| Hexapeptide-19 (and) | |
| Dextrane (and) Acrylates | |
| Copolymer (and) Carbomer | |
| (and) Phenoxyethanol | |
| Glycerin (and) Water (and) | 0-30.00% |
| Myristoyl Octapeptide-1 | |
| Hexapeptide-10 | 0-30.00% |
| Glutamylamidoethyl Indol | 0-10.00% |
| (and) Water | |
| Butylene Glycol (and) | 0-10.00% |
| Pyroglutamylamidoethyl | |
| Indole (and) Water | |
| Dipropylene Glycol (and) | 0-5.00% |
| Quercus Suber Bark Extract | |
| Silica (and) | 0-5.00% |
| DMPA/Isophthalic | |
| Acid/SMDI Copolymer | |
| Cyclopentasiloxane (and) | 0-10.00% |
| Dimethiconol (and) | |
| Ethylhexyl Cocoate (and) | |
| Dimethylmethoxy Chromanyl | |
| Palmitate | |
| Butylene Glycol (and) Water | 0-10.00% |
| (and) Laureth-3 (and) | |
| Hydroxyethylcellulose (and) | |
| Acetyl Dipeptide-1 Cetyl | |
| Ester | |
| Propylene Glycol (and) | 0-2.00% |
| Diazolidinyl Urea (and) | |
| Methylparaben (and) | |
| Propylparaben | |
| Sodium Hydroxide | 0-5.00% |
| Total | 100% |
| DEIONIZED WATER | QS |
| GLYCERIN | 0-10.00% |
| HISTIDINE HCL | 0-0.50% |
| DL PANTHENOL | 0-5.00% |
| METHYLPARABEN | 0-0.50% |
| PROTAMATE 4400 DPS | 0-5.00% |
| FINSOLV TN | 0-5.00% |
| GMS 450 | 0-5.00% |
| AVOCADO OIL | 0-5.00% |
| ISOCETYL STEARATE | 0-5.00% |
| (DERMOL ICS) | |
| VITAMIN E ACETATE | 0-5.00% |
| CETYL ALCOHOL | 0-5.00% |
| DC 200/100 | 0-5.00% |
| LANOLIN OIL | 0-5.00% |
| PGMS | 0-5.00% |
| SILICONE 580 WAX | 0-5.00% |
| VITAMIN A PALMITATE | 0-5.00% |
| BISABOLOL | 0-5.00% |
| LANOLIN ALCOHOL | 0-5.00% |
| PROPYLPARABEN | 0-0.50% |
| GERMALL II | 0-0.50% |
| Acetyl Hexapeptide-25 | 0-10.00% |
| Water (and) Palmitoyl | 0-10.00% |
| Hexapeptide-19 (and) | |
| Dextrane (and) Acrylates | |
| Copolymer (and) Carbomer | |
| (and) Phenoxyethanol | |
| Glycerin (and) Water (and) | 0-30.00% |
| Myristoyl Octapeptide-1 | |
| Hexapeptide-10 | 0-30.00% |
| Glutamylamidoethyl Indol | 0-10.00% |
| (and) Water | |
| Butylene Glycol (and) | 0-10.00% |
| Pyroglutamylamidoethyl | |
| Indole (and) Water | |
| Dipropylene Glycol (and) | 0-5.00% |
| Quercus Suber Bark Extract | |
| Silica (and) | 0-5.00% |
| DMPA/Isophthalic | |
| Acid/SMDI Copolymer | |
| Cyclopentasiloxane (and) | 0-10.00% |
| Dimethiconol (and) | |
| Ethylhexyl Cocoate (and) | |
| Dimethylmethoxy Chromanyl | |
| Palmitate | |
| Butylene Glycol (and) Water | 0-10.00% |
| (and) Laureth-3 (and) | |
| Hydroxyethylcellulose (and) | |
| Acetyl Dipeptide-1 Cetyl Ester | |
| Total | 100% |
| DI WATER | QS |
| DISODIUM EDTA | 0-0.50% |
| ALLANTOIN | 0-5.00% |
| DL-PANTHENOL | 0-5.00% |
| CARBOPOL 940 | 0-2.00% |
| EMCOL E607 | 0-2.00% |
| SHEA BUTTER | 0-5.00% |
| DC 200 FLUID | 0-5.00% |
| SQUALANE | 0-5.00% |
| WHITE BEESWAX | 0-5.00% |
| PGMS | 0-5.00% |
| NEOBEE M5 | 0-5.00% |
| CETYL ALCOHOL | 0-5.00% |
| GMS 450 | 0-5.00% |
| CERAMIDES III | 0-1.00% |
| CREMOPHOR RH 40 | 0-5.00% |
| (BASF) | |
| EMERSOL 315 | 0-5.00% |
| OLEIC ACID (EMERSOL | 0-5.00% |
| 221) | |
| VITAMIN A PALMITATE | 0-5.00% |
| ASORBOSILANE C | 0-5.00% |
| VITAMIN E ACETATE | 0-5.00% |
| TEA | 0-2.00% |
| MATRIXYL 3000 | 0-10.00% |
| ALOE VERA GEL | 0-5.00% |
| BIOGEN SEA COMPLEX | 0-5.00% |
| COLLASOL M | 0-5.00% |
| AVOCADO OIL | 0-5.00% |
| ROSE HIP OIL | 0-5.00% |
| PANSY EXTRACT | 0-5.00% |
| FENNEL SEED EXTRACT | 0-5.00% |
| Acetyl Hexapeptide-25 | 0-10.00% |
| Water (and) Palmitoyl | 0-10.00% |
| Hexapeptide-19 (and) | |
| Dextrane (and) Acrylates | |
| Copolymer (and) Carbomer | |
| (and) Phenoxyethanol | |
| Glycerin (and) Water (and) | 0-30.00% |
| Myristoyl Octapeptide-1 | |
| Hexapeptide-10 | 0-30.00% |
| Glutamylamidoethyl Indol | 0-10.00% |
| (and) Water | |
| Butylene Glycol (and) | 0-10.00% |
| Pyroglutamylamidoethyl | |
| Indole (and) Water | |
| Dipropylene Glycol (and) | 0-5.00% |
| Quercus Suber Bark Extract | |
| Silica (and) DMPA/Isophthalic | 0-5.00% |
| Acid/SMDI Copolymer | |
| Cyclopentasiloxane (and) | 0-10.00% |
| Dimethiconol (and) Ethylhexyl | |
| Cocoate (and) | |
| Dimethylmethoxy Chromanyl | |
| Palmitate | |
| Butylene Glycol (and) Water | 0-10.00% |
| (and) Laureth-3 (and) | |
| Hydroxyethylcellulose (and) | |
| Acetyl Dipeptide-1 Cetyl Ester | |
| DOWICIL 200 | 0-1.00% |
| GERMABEN II | 0-2.00% |
| BIOCELL SOD | 0-5.00% |
| SEPIGEL 305 | 0-3.00% |
| Total | 100% |
| Water | QS |
| Acrylates/C10-30 Alkyl | 0-2.00% |
| Acrylate Crosspolymer | |
| Propylene Glycol | 0-5.00% |
| Allantoin | 0-5.00% |
| Caprylic/Capric Triglyceride | 0-5.00% |
| C12-15 Alkyl Benzoate | 0-5.00% |
| Glyceryl Stearate, PEG-100 | 0-5.00% |
| Stearate | |
| Cetyl Alcohol | 0-5.00% |
| Dimethicone | 0-5.00% |
| Stearic Acid | 0-5.00% |
| Octisalate | 0-5.00% |
| Octinoate | 0-7.50% |
| Oxylbenzone | 0-4.00% |
| Titanium Dioxide, Dimethicone | 0-1.00% |
| Triethanolamine | 0-2.00% |
| Acetyl Hexapeptide-25 | 0-10.00% |
| Water (and) Palmitoyl | 0-10.00% |
| Hexapeptide-19 (and) Dextrane | |
| (and) Acrylates Copolymer | |
| (and) Carbomer (and) | |
| Phenoxyethanol | |
| Glycerin (and) Water (and) | 0-30.00% |
| Myristoyl Octapeptide-1 | |
| Hexapeptide-10 | 0-30.00% |
| Glutamylamidoethyl Indol (and) | 0-10.00% |
| Water | |
| Butylene Glycol (and) | 0-10.00% |
| Pyroglutamylamidoethyl Indole | |
| (and) Water | |
| Dipropylene Glycol (and) | 0-5.00% |
| Quercus Suber Bark Extract | |
| Silica (and) DMPA/Isophthalic | 0-5.00% |
| Acid/SMDI Copolymer | |
| Cyclopentasiloxane (and) | 0-10.00% |
| Dimethiconol (and) Ethylhexyl | |
| Cocoate (and) | |
| Dimethylmethoxy Chromanyl | |
| Palmitate | |
| Butylene Glycol (and) Water | 0-10.00% |
| (and) Laureth-3 (and) | |
| Hydroxyethylcellulose (and) | |
| Acetyl Dipeptide-1 Cetyl Ester | |
| Total | 100% |

SILICONE-IN-WATER EMULSION

| Ingredient | % w/w |
|---|---|
| Water | QS |
| Cyclopentasiloxane | 0-5.00% |
| Squalane | 0-7.00% |
| Caprylic/Capric | 0-15.00% |
| Triglyceride | |
| Glycerin (and) Water (and) | 0-10.00% |
| Urea (and) Sodium PCA | |
| (and) Trehalose (and) | |
| Polyquaternium-51 (and) | |
| Triacetin (and) Sodium | |
| Hyaluronate | |
| Hydrolyzed Oats | 0-0.50% |
| Butylene Glycol (and) | 0-10.00% |
| Water (and) Laureth-3 (and) | |
| Hydroxyethylcellulose (and) | |
| Acetyl Dipeptide-1 Cetyl | |
| Ester | |
| Acetyl Hexapeptide-25 | 0-10.00% |
| Water (and) Phospholipids | 0-5.00% |
| (and) Tocopheryl Acetate | |
| (and) Retinyl Palmitate | |
| (and) Ascorbyl Palmitate | |
| Cyclopentasiloxane (and) | 0-10.00% |
| Dimethiconol (and) | |
| Ethylhexyl Cocoate (and) | |
| Dimethylmethoxy | |
| Chromanyl Palmitate | |
| Dimethicone (and) | 0-10.00% |
| Cyclopentasiloxane (and) | |
| Polysilicone-11 (and) | |
| Nylon-12 (and) Silica (and) | |
| PEG-10 Dimethicone (and) | |
| Polysorbate 40 (and) | |
| Isohexadecane (and) | |
| Ammonium | |
| Polyacryloyldimethyl | |
| Taurate | |
| Butylene Glycol (and) | 0-10.00% |
| Pyroglutamylamidoethyl | |
| Indole (and) Water | |
| Mica | 0-1.00% |
| Silica (and) | 0-5.00% |
| DMPA/Isophthalic | |
| Acid/SMDI Copolymer | |
| Sodium Acrylate/Sodium | 0-3.00% |
| Acryloyldimethyl Taurate | |
| Copolymer (and) | |
| Isohexadecane (and) | |
| Polysorbate 80 | |
| Hexapeptide-10 | 0-30.00% |
| Glycerin (and) Water (and) | 0-30.00% |
| Myristoyl Octapeptide-1 | |
| Water (and) Palmitoyl | 0-10.00% |
| Hexapeptide-19 (and) | |
| Dextrane (and) Acrylates | |
| Copolymer (and) Carbomer | |
| (and) Phenoxyethanol | |
| Glutamylamidoethyl Indol | 0-10.00% |
| (and) Water | |
| Dipropylene Glycol (and) | 0-5.00% |
| Quercus Suber Bark Extract | |
| Propylene Glycol (and) | 0-2.00% |
| Diazolidinyl Urea (and) | |
| Methylparaben (and) | |
| Propylparaben | |
| Total | 100.00% |

HYDRO-GEL

| Ingredient | % w/w |
|---|---|
| Water | QS |
| Carbomer | 0-2.00% |
| Panthenol | 0-5.00% |
| Butylene Glycol | 0-10.00% |
| Xanthan Gum | 0-2.00% |
| Bis-PEG-18 Methyl Ether | 0-5.00% |
| Dimethyl Silane | |
| Dimethicone PEG-7 Phosphate | 0-5.00% |
| Squalane | 0-5.00% |
| PEG-75 | 0-5.00% |
| Acetyl Hexapeptide-25 | 0-10.00% |
| Water (and) Palmitoyl | 0-10.00% |
| Hexapeptide-19 (and) Dextrane | |
| (and) Acrylates Copolymer (and) | |
| Carbomer (and) Phenoxyethanol | |
| Glycerin (and) Water (and) | 0-30.00% |
| Myristoyl Octapeptide-1 | |
| Hexapeptide-10 | 0-30.00% |
| Glutamylamidoethyl Indol (and) | 0-10.00% |
| Water | |
| Butylene Glycol (and) | 0-10.00% |
| Pyroglutamylamidoethyl Indole | |
| (and) Water | |
| Dipropylene Glycol (and) | 0-5.00% |
| Quercus Suber Bark Extract | |
| Silica (and) DMPA/Isophthalic | 0-5.00% |
| Acid/SMDI Copolymer | |
| Cyclopentasiloxane (and) | 0-10.00% |
| Dimethiconol (and) Ethylhexyl | |
| Cocoate (and) Dimethylmethoxy | |
| Chromanyl Palmitate | |
| Butylene Glycol (and) Water | 0-10.00% |
| (and) Laureth-3 (and) | |
| Hydroxyethylcellulose (and) | |
| Acetyl Dipeptide-1 Cetyl Ester | |
| Propylene Glycol (and) | 0-2.00% |
| Diazolidinyl Urea, | |
| Methylparaben, Propylparaben | |
| Hydrogenated Soybean Oil (and) | 0-5.00% |
| Vanilla Planifolia Fruit Extract | |
| Caprylic/Capric Triglyceride | 0-5.00% |
| (and) Citrus Medica Limonum | |
| (Lemon) Peel Extract (and) | |
| Zingiber Officinale (Ginger) | |
| Root Extract | |
| Rosa Centifolia Flower Water | 0-20.00% |
| Red 33 | 0-0.50% |
| Yellow 5 | 0-0.50% |
| Total | 100.00% |
| Water | QS |
| Acrylates/C10-C29 Alkyl | 0-2.00% |
| Acrylate Crosspolymer | |
| Aloe Barbadensis Leaf Juice | 0-2.00% |
| C4-24 Alkyl | 0-10.00% |
| Dimethicone/divinyldimethicone | |
| crosspolymer cyclomethicone | |
| Dimethicone/Divinyldimethicone | 0-5.00% |
| Crosspolymer Dimethicone | |
| C4-24 Alkyl Dimethicone/ | 0-5.00% |
| Divinyldimethicone | |
| Crosspolymer Isononanoate | |
| RESSASSOL VS | 0-5.00% |
| Acetyl Hexapeptide-25 | 0-10.00% |
| Water (and) Palmitoyl | 0-10.00% |
| Hexapeptide-19 (and) Dextrane | |
| (and) Acrylates Copolymer (and) | |
| Carbomer (and) Phenoxyethanol | |
| Glycerin (and) Water (and) | 0-30.00% |
| Myristoyl Octapeptide-1 | |
| Hexapeptide-10 | 0-30.00% |
| Glutamylamidoethyl Indol (and) | 0-10.00% |
| Water | |
| Butylene Glycol (and) | 0-10.00% |
| Pyroglutamylamidoethyl Indole | |
| (and) Water | |
| Dipropylene Glycol (and) | 0-5.00% |
| Quercus Suber Bark Extract | |
| Silica (and) DMPA/Isophthalic | 0-5.00% |
| Acid/SMDI Copolymer | |
| Cyclopentasiloxane (and) | 0-10.00% |
| Dimethiconol (and) Ethylhexyl | |
| Cocoate (and) Dimethylmethoxy | |
| Chromanyl Palmitate | |
| Butylene Glycol (and) Water | 0-10.00% |
| (and) Laureth-3 (and) | |
| Hydroxyethylcellulose (and) | |
| Acetyl Dipeptide-1 Cetyl Ester | |
| Prunus Amygdalus Dulis (Sweet | 0-5.00% |
| Almond) Oil | |
| Glycosamines | 0-5.00% |
| Caprylyl Glycol (and) | 0-5.00% |
| Phenoxyethanol | |
| Sodium Hydroxide | 0-2.00% |
| Total | 100.00% |
| DEIONIZED WATER | QS |
| CARBOPOL 940 | 0-2.00% |
| BUTYLENE GLYCOL | 0-5.00% |
| ALOE 200M | 0-2.00% |
| GLYCERIN | 0-5.00% |
| KESSCO OP | 0-20.00% |
| ALMOND OIL | 0-10.00% |
| VITAMIN E ACETATE | 0-5.00% |
| PROCOL CS-20D | 0-5.00% |
| CETYL ALCOHOL | 0-5.00% |
| Acetyl Hexapeptide-25 | 0-10.00% |
| Water (and) Palmitoyl | 0-10.00% |
| Hexapeptide-19 (and) Dextrane | |
| (and) Acrylates Copolymer (and) | |
| Carbomer (and) Phenoxyethanol | |
| Glycerin (and) Water (and) | 0-30.00% |
| Myristoyl Octapeptide-1 | |
| Hexapeptide-10 | 0-30.00% |
| Glutamylamidoethyl Indol (and) | 0-10.00% |
| Water | |
| Butylene Glycol (and) | 0-10.00% |
| Pyroglutamylamidoethyl Indole | |
| (and) Water | |
| Dipropylene Glycol (and) | 0-5.00% |
| Quercus Suber Bark Extract | |
| Silica (and) DMPA/Isophthalic | 0-5.00% |
| Acid/SMDI Copolymer | |
| Cyclopentasiloxane (and) | 0-10.00% |
| Dimethiconol (and) Ethylhexyl | |
| Cocoate (and) Dimethylmethoxy | |
| Chromanyl Palmitate | |
| Butylene Glycol (and) Water | 0-10.00% |
| (and) Laureth-3 (and) | |
| Hydroxyethylcellulose (and) | |
| Acetyl Dipeptide-1 Cetyl Ester | |
| Total | 100.00% |
| Water | QS |
| Hydroxyethylcellulose | 0-2.00% |
| Acetyl Hexapeptide-25 | 0-10.00% |
| Water (and) Palmitoyl | 0-10.00% |
| Hexapeptide-19 (and) Dextrane | |
| (and) Acrylates Copolymer (and) | |
| Carbomer (and) Phenoxyethanol | |
| Glycerin (and) Water (and) | 0-30.00% |
| Myristoyl Octapeptide-1 | |
| Hexapeptide-10 | 0-30.00% |
| Glutamylamidoethyl Indol (and) | 0-10.00% |
| Water | |
| Butylene Glycol (and) | 0-10.00% |
| Pyroglutamylamidoethyl Indole | |
| (and) Water | |
| Dipropylene Glycol (and) | 0-5.00% |
| Quercus Suber Bark Extract | |
| Silica (and) DMPA/Isophthalic | 0-5.00% |
| Acid/SMDI Copolymer | |
| Cyclopentasiloxane (and) | 0-10.00% |
| Dimethiconol (and) Ethylhexyl | |
| Cocoate (and) Dimethylmethoxy | |
| Chromanyl Palmitate | |
| Butylene Glycol (and) Water | 0-10.00% |
| (and) Laureth-3 (and) | |
| Hydroxyethylcellulose (and) | |
| Acetyl Dipeptide-1 Cetyl Ester | |
| Pentylene Glycol | 0-5.00% |
| Lipolight OAP | 0-5.00% |
| Disodium EDTA | 0-0.50% |
| Phenoxyethanol (and) Caprylyl | 0-2.00% |
| Glycol (and) Ethylhexylglycerin | |
| (and) Hexylene Glycol | |
| Water (Aqua) (and) | 0-10.00% |
| Hexapeptide-10 | |
| PEG-8 Dimethicone | 0-5.00% |
| Sensitive Skin Maskant 905613 | 0-2.00% |
| Lactobacillus/Eriodictyon | 0-5.00% |
| Californicum Ferment Extract | |
| Ethanol | 0-5.00% |
| Phenylethyl Resorcinol | 0-5.00% |
| Citric Acid | 0-2.00% |
| Total | 100% |
| Water | QS |
| Carbomer | 0-2.00% |
| Methyl Gluceth-20 | 0-5.00% |
| Allantoin | 0-5.00% |
| Aloe Barbadensis Leaf Juice | 0-5.00% |
| Disodium EDTA | 0-0.50 |
| Glycerin (and) Water (and). | 0-10.00% |
| Sodium Polyacrylate | |
| Trietholamine | 0-2.00% |
| C12-15 Alkyl Benzoate | 0-5.00% |
| Glycerin (and) Water (and) | 0-10.00% |
| Butylene Glycol (and) Carbomer | |
| (and) Polysorbate 20 (and) | |
| Palmitoyl Oligopeptide (and) | |
| Palmitoyl Tetrapeptide-7 | |
| Cyclomethicone | 0-5.00% |
| Dimethicone | 0-5.00% |
| Acetyl Hexapeptide-25 | 0-10.00% |
| Water (and) Palmitoyl | 0-10.00% |
| Hexapeptide-19 (and) Dextrane | |
| (and) Acrylates Copolymer (and) | |
| Carbomer (and) Phenoxyethanol | |
| Glycerin (and) Water (and) | 0-30.00% |
| Myristoyl Octapeptide-1 | |
| Hexapeptide-10 | 0-30.00% |
| Glutamylamidoethyl Indol (and) | 0-10.00% |
| Water | |
| Butylene Glycol (and) | 0-10.00% |
| Pyroglutamylamidoethyl Indole | |
| (and) Water | |
| Dipropylene Glycol (and) | 0-5.00% |
| Quercus Suber Bark Extract | |
| Silica (and) DMPA/Isophthalic | 0-5.00% |
| Acid/SMDI Copolymer | |
| Cyclopentasiloxane (and) | 0-10.00% |
| Dimethiconol (and) Ethylhexyl | |
| Cocoate (and) Dimethylmethoxy | |
| Chromanyl Palmitate | |
| Butylene Glycol (and) Water | 0-10.00% |
| (and) Laureth-3 (and) | |
| Hydroxyethylcellulose (and) | |
| Acetyl Dipeptide-1 Cetyl Ester | |
| Phenoxyethanol (and) | 0-2.00% |
| Methylparaben (and) Ethylparaben | |
| (and) Butylparaben (and) | |
| Propylparaben (and) Isobutylparab | |
| Total | 100.00% |

AQUEOUS SERUM

| Ingredient | % w/w |
|---|---|
| Water | QS |
| AH CARE SOLUTION | 0-10.00% |
| Chamomile Extract | 0-5.00% |
| Green Tea Extract | 0-5.00% |
| Comfrey Extract | 0-5.00% |
| Butylene Glycol | 0-5.00% |
| Acetyl Hexapeptide-25 | 0-10.00% |
| Water (and) Palmitoyl | 0-10.00% |
| Hexapeptide-19 (and) Dextrane | |
| (and) Acrylates Copolymer | |
| (and) Carbomer (and) | |
| Phenoxyethanol | |
| Glycerin (and) Water (and) | 0-30.00% |
| Myristoyl Octapeptide-1 | |
| Hexapeptide-10 | 0-30.00% |
| Glutamylamidoethyl Indol (and) | 0-10.00% |
| Water | |
| Butylene Glycol (and) | 0-10.00% |
| Pyroglutamylamidoethyl Indole | |
| (and) Water | |
| Dipropylene Glycol (and) | 0-5.00% |
| Quercus Suber Bark Extract | |
| Silica (and) DMPA/Isophthalic | 0-5.00% |
| Acid/SMDI Copolymer | |
| Cyclopentasiloxane (and) | 0-10.00% |
| Dimethiconol (and) Ethyihexyl | |
| Cocoate (and) | |
| Dimethylmethoxy Chromanyl | |
| Palmitate | |
| Butylene Glycol (and) Water | 0-10.00% |
| (and) Laureth-3 (and) | |
| Hydroxyethylcellulose (and) | |
| Acetyl Dipeptide-1 Cetyl Ester | |
| Germaben II | 0-2.00% |
| Vitamin E | 0-5.00% |
| Vitamin C(Stay C-50) | 0-5.00% |
| Vitamin A Palmitate 1.0 m | 0-5.00% |
| I.U./g and Vitamin D3 100,000 | |
| I.U./g | |
| ROSE WATER | 0-10.00% |
| Witch Hazel | 0-10.00% |
| Sodium Hydroxide 50% soln. | 0-7.00% |

### EXAMPLE 2

A composition that can be topically applied by a human as a cream emulsion includes the following ingredients in three phases:

| | | concentration | |
|---|---|---|---|
| phase | component | (by weight) | function and source |
| water-based phase | Water | QS | skin moisturizer |
| water-based phase | Squalane | 0-10.00% | skin moisturizer |
| water-based phase | Cyclopentasiloxane | 0-5.00% | skin moisturizer |
| water-based phase | Caprylic/Capric | 0-13.00% | skin moisturizer |
| | Triglyceride | | |
| water-based phase | Glycerin (and) Water | 0-10.50% | skin moisturizer |
| | (and) Urea (and) Sodium | | |
| | PCA (and) Trehalose | | |
| | (and) Polyquaternium-51 | | |
| | (and) Triacetin (and) | | |
| | Sodium Hyaluronate | | |
| water-based phase | Hydrolyzed Oats | 0-1.00% | skin moisturizer |
| water-based phase | Hexapeptide-10 | 0-20.00% | Improves cell adhesion, |
| | | | increases cell proliferation, |
| | | | increases dermis density |
| water-based phase | Glycerin (and) Water | 0-20.00% | Stimulates cell replication and |
| | (and) Myristoyl | | collagen |
| | Octapeptide-1 | | |
| water-based phase | Water (and) Palmitoyl | 0-10.00% | Inhibits Acetylcholine release |
| | Hexapeptide-19 (and) | | |
| | Dextrane (and) Acrylates | | |
| | Copolymer (and) | | |
| | Carbomer (and) | | |
| | Phenoxyethanol | | |
| water-based phase | Glutamylamidoethyl Indol | 0-5.00% | Cutaneous neuroprotection |
| | (and) Water | | |
| water-based phase | Butylene Glycol (and) | 0-5.00% | Cutaneous neuroprotection |
| | Pyroglutamylamidoethyl | | |
| | Indole (and) Water | | |
| water-based phase | Dipropylene Glycol (and) | 0-1.00% | Improves skin roughness |
| | Quercus Suber Bark | | through tighetening effect |
| | Extract | | |
| water-based phase | Butylene Glycol (and) | 0-20.00% | Inhibits muscle contractions in |
| | Water (and) Laureth-3 | | skin |
| | (and) | | |
| | Hydroxyethylcellulose | | |
| | (and) Acetyl Dipeptide-1 | | |
| | Cetyl Ester | | |
| water-based phase | Acetyl Hexapeptide-25 | 0-8.00% | Inhibition of acetylcholine |
| | | | receptor clustering |
| water-based phase | Water (and) | 0-2.00% | Vitamins |
| | Phospholipids (and) | | |
| | Tocopheryl Acetate (and) | | |
| | Retinyl Palmitate (and) | | |
| | Ascorbyl Palmitate | | |
| water-based phase | Cyclopentasiloxane (and) | 0-15.00% | Inhibits tyrosinase activity |
| | Dimethiconol (and) | | |
| | Ethylhexyl Cocoate (and) | | |
| | Dimethylmethoxy | | |
| | Chromanyl Palmitate | | |
| water-based phase | Propylene Glycol (and) | 0-2.00% | Preservative |
| | Diazolidinyl Urea (and) | | |
| | Methylparaben (and) | | |
| | Propylparaben | | |
| silicone-based | Dimethicone (and) | 0-10.00% | Feel modifier |
| phase | Cyclopentasiloxane (and) | | |
| | Polysilicone-11 (and) | | |
| | Nylon-12 (and) Silica | | |
| | (and) PEG-10 | | |
| | Dimethicone (and) | | |
| | Polysorbate 40 (and) | | |
| | Isohexadecane (and) | | |
| | Ammonium | | |
| | Polyacryloyldimethyl | | |
| | Taurate | | |
| silicone-based | Mica | 0-1.00% | Light diffuser |
| phase | | | |
| silicone-based | Silica (and) | 0-5.00% | Light diffuser |
| phase | DMPA/Isophthalic | | |
| | Acid/SMDI Copolymer | | |
| Polymer-based | Sodium Acrylate/Sodium | 0-3.00% | Thickening agent |
| phase | Acryloyldimethyl Taurate | | |
| | Copolymer (and) | | |
| | Isohexadecane (and) | | |
| | Polysorbate 80 | | |

### Manufacturing Procedure:

Charge water into main tank with propeller mixing. A vortex should form. Charge the remaining water-based phase ingredients individually and mix. Slowly sift in silicone-based phase ingredients individually and mix until homogenous. Charge polymer-based phase and mix well until homogenous. If necessary, homogenize batch to attain a smooth consistency.

In describing embodiments of the disclosure, specific terminology is used for the sake of clarity. For purposes of description, each specific term is intended to at least include all technical and functional equivalents that operate in a similar manner to accomplish a similar purpose. Additionally, in some instances where a particular embodiment of the disclosure includes a plurality of elements or method steps, those elements or steps may be replaced with a single element or step; likewise, a single element or step may be replaced with a plurality of elements or steps that serve the same purpose. Moreover, while this disclosure has been shown and described with references to particular embodiments thereof, those skilled in the art will understand that various other changes in form and details may be made therein without departing from the scope of the disclosure.

All publications and patent applications cited in this specification are herein incorporated by reference as if each individual publication or patent application were specifically and individually indicated to be incorporated by reference.

Although the foregoing disclosure has been described in some detail by way of illustration and example for purposes of clarity of understanding, it will be readily apparent to those of ordinary skill in the art in light of the teachings of this disclosure that certain changes and modifications may be made thereto without departing from the spirit or scope of the appended claims.

What is claimed is:

1. A cosmetic composition for topical application to the skin, comprising as active agents:
a. at least one proliferation promoting peptide selected from the group consisting of: a hexapeptide-10, an Myristoyl Octapeptide-1, and an acetyl dipeptide-1 cetyl ester;
b. at least one neuro-enhancing peptide selected from the group consisting of: a hexapeptide-19, a hexapeptide-25, a proglutamylamidoethyl indole, and a glutamylamidoethyl indole;
c. at least one skin appearance enhancing factor selected from the group consisting of: a quercus suber bark extract, a dimethyl chromanyl palmitate, and silica in addition with DMPA/Isophthalic acid/SMDI copolymer; and a cosmetically acceptable vehicle.

2. The cosmetic composition of claim 1, wherein the composition comprises at least two members of the group consisting of a hexapeptide-10, an Myristoyl Octapeptide-1, and an acetyl dipeptide-1 cetyl ester; at least two members from the group consisting of: a hexapeptide-19, a hexapeptide-25, a proglutamylamidoethyl indole, and a glutamylamidoethyl indole; and at least two members from the group consisting of: a quercus suber bark extract, a dimethyl chromanyl palmitate, and silica in addition with DMPA/Isophthalic acid/SMDI copolymer.

3. The cosmetic composition of claim 2, wherein the composition comprises a hexapeptide-10, an Myristoyl Octapeptide-1, an acetyl dipeptide-1 cetyl ester, a hexapeptide-19, a hexapeptide-25, a glutamylamidoethyl indole, a quercus suber bark extract, a dimethyl chromanyl palmitate, and silica in addition with DMPA/Isophthalic acid/SMDI copolymer.

4. The cosmetic composition of claim 3, wherein the:
a. proliferation promoting peptide(s) is present in an amount that ranges from about 0.001 to about 33% by weight;
b. neuro-enhancing peptide(s) is present in an amount that ranges from about 0.001 to about 33% by weight; and
c. skin appearance enhancing factor(s) is present in an amount that ranges from about 0.001 to about 20% by weight.

5. The cosmetic composition of claim 3, further comprising a lipophilic agent.

6. The cosmetic composition of claim 3, further comprising a surfactant.

7. The cosmetic composition of claim 3, wherein the pH of said composition is in the range of about a 3 to about a 8.

8. The cosmetic composition of claim 7, wherein the pH of said composition is in the range of about a 4.5 to about a 6.5.

9. The cosmetic composition of claim 3, wherein said composition is formulated for application to the skin of the face; surrounding the eye; lips; surrounding the lips;
forehead; neck; hands; fingers; feet; toes; skin of the joints; under arms; and body.

10. The cosmetic composition of claim 3, wherein said cosmetically acceptable vehicle is an oil in water, water in oil emulsion, water in silicone, silicone in water, and a hydrogel, aqueous alcoholic.

11. The cosmetic composition of claim 3, wherein said cosmetically acceptable carrier is a member selected from the group consisting of glycerin, butylene glycol, propylene glycol, water, jojoba oil, sweet almond oil, soybean oil, sunflower oil, and apricot oil.

12. The cosmetic composition of claim 3, wherein said composition further comprises a sunblock.

13. A method for conditioning the skin, the method comprising topically applying a cosmetic composition comprising:
a. at least one proliferation promoting peptide selected from the group consisting of: a hexapeptide-10, an Myristoyl Octapeptide-1, and an acetyl dipeptide-1 cetyl ester;
b. at least one neuro-enhancing peptide selected from the group consisting of: a hexapeptide-19, a hexapeptide-25, a proglutamylamidoethyl indole, and a glutamylamidoethyl indole;
c. at least one skin appearance enhancing factor selected from the group consisting of a quercus suber bark extract, a dimethyl chromanyl palmitate, an silica in addition with DMPA/Isophthalic acid/SMDI copolymer; and a cosmetically acceptable vehicle.

14. The method of claim 13, wherein the cosmetic composition comprises at least two members of the group consisting of a hexapeptide-10, an Myristoyl Octapeptide-1, and an acetyl dipeptide-1 cetyl ester; at least two members from the group consisting of: a hexapeptide-19, a hexapeptide-25, a proglutamylamidoethyl indole, and a glutamylamidoethyl indole; and at least two members from the group consisting of: a quercus suber bark extract, a dimethyl chromanyl palmitate, and silica in addition with DMPA/Isophthalic acid/SMDI copolymer.

15. The method of claim 14, wherein the cosmetic composition comprises a hexapeptide-10, an Myristoyl Octapeptide-1, an acetyl dipeptide-1 cetyl ester, a hexapeptide-19, a hexapeptide-25, a glutamylamidoethyl indole, a quercus suber bark extract, a dimethyl chromanyl palmitate, and silica in addition with DMPA/Isophthalic acid/SMDI copolymer.

16. The method of claim 15, wherein the:
a. proliferation promoting peptide(s) is present in an amount that ranges from about 0.001 to about 33% by weight;
b. neuro-enhancing peptide(s) is present in an amount that ranges from about 0.001 to about 33% by weight; and
c. skin appearance enhancing factor(s) is present in an amount that ranges from about 0.001 to about 20% by weight.

17. The method of claim 15, further comprising applying said composition to the skin around the eyes, mouth, lips, eyelids, eyebrows, nose, ears, neck, hands, feet, fingers, toes, and overall body.
